(19) United States
(12) Patent Application Publication
(10) Pub. No.: US 2006/0111676 A1
(43) Pub. Date: May 25, 2006

(54) Passively guarded, pre-filled injection syringe

(76) Inventor: Don Millerd, San Diego, CA (US)

Correspondence Address: Attn: Neil K. Nydegger;NYDEGGER & ASSOCIATES, 348 Olive Street, San Diego, CA, 92103, US

(21) Appl. No.: 11/140,583

(22) Filed: May 27, 2005

Related U.S. Application Data
(63) Continuation-in-part of application No. 11/055,415, filed on Feb. 10, 2005.
Application No. 11/055,415 is a continuation-in-part of application No. 10/983,108, filed on Nov. 5, 2004.

(51) Int. Cl. A61M 5/00

(52) U.S. Cl. 604/187

(57) ABSTRACT

A fluid transfer apparatus for use with an inverted syringe includes: a pre-filled vial that is mounted on a housing, and a needle assembly that is mounted on the housing for movement between first and second positions. In the first position, a double-ended needle in the assembly is poised on the housing to penetrate a bung to thereby establish fluid communication with fluid in the vial. When moved to the second position, fluid communication is established, and a flange on the needle assembly engages with the housing to fixedly hold the needle in its second position.

This application is a continuation-in-part of application Ser. No. 11/055,415, filed Feb. 10, 2005, which is currently pending, and which is a continuation-in-part of application Ser. No. 10/983,108, filed Nov. 5, 2004, which is currently pending. The contents of application Ser. No. 11/055,415 and application Ser. No. 10/983,108 are incorporated herein by reference.

### FIELD OF THE INVENTION

The present invention pertains generally to pre-filled syringes. More particularly, the present invention pertains to a fluid transfer apparatus and to methods that maintain the integrity of a pre-filled syringe prior to its use. The present invention is particularly, but not exclusively useful, as a fluid transfer apparatus for an inverted syringe that incorporates a pre-filled fluid container (vial).

### BACKGROUND OF THE INVENTION

Recent research from the Centers for Disease Control and Prevention (CDC) shows that approximately 384,000 needle sticks or similar injuries occur among health care workers in U.S. hospitals each year. Unfortunately, each accidental needle stick has the potential to expose a health care worker to a life-threatening virus such as hepatitis or HIV. In addition to the needle sticks that occur in hospitals, accidental needle sticks can also occur in other health care settings. For example, needle stick injuries can occur at clinics or during home health care. In fact, some studies have estimated that over 600,000 needle sticks occur in the U.S. each year, and approximately 1,000 of these accidental needle sticks result in a life-threatening infection.

For each accidental needle stick, health care providers are obligated to test and counsel the exposed worker. Further, follow-up testing for HIV must be conducted approximately six months after the exposure. It is to be appreciated that the costs associated with the testing, lab work, the worker's lost time, and the associated tracking and administrative costs, can be considerable.

Accidental needle sticks can occur in several ways. For example, sudden movement by the patient can cause a health care worker to lose control of a syringe, resulting in injury. Attempts to manually recap a needle following an injection can also result in injury. Moreover, injuries often result when contaminated, unprotected needles are left unattended or disposed of improperly. In addition to accidental needle sticks, unnecessary exposure to bloodborne pathogens can result when a health care worker mistakenly reuses a contaminated needle on a patient.

One particular type of syringe that is prone to needle stick injuries is the fillable injection syringe. In overview, these fillable injection syringes are designed to be filled with a medicament from a medicament vial by the same user that administers an injection. Heretofore, a typical procedure has involved removing a cap that covers the sharp needle tip of the fillable injection syringe. With the needle exposed, the needle tip is inserted into a vial containing medicament. This step generally occurs just prior to an injection. Next, the plunger is depressed to void the syringe chamber of air. With the syringe voided, the plunger is retracted to draw a specified quantity of medicament into the syringe chamber. Once the medicament has been loaded into the syringe, the needle is then inserted into a patient and the plunger is depressed to inject the medicament into the patient. After the injection, the needle is removed from the patient and often must be manually recapped to protect the contaminated needle. After recapping, it is often difficult to distinguish between used and unused syringes.

Fillable injection syringes and needles are often obtained separately. Typically, the syringes are available for use with different sized needles. This allows doctors to obtain and store fewer syringes. Then, when an injection is needed, a desired needle is simply mounted on a syringe.

As is well known, pre-filled syringes are useful and, indeed, are sometimes preferred for certain applications. When a pre-filled syringe is to be used, however, it is always important that the fluid medicament be somehow properly maintained until it is to be actually injected. One way to do this is to confine the fluid in a fluid-tight chamber. Such chambers, however, must eventually be accessible for fluid transfer. Thus, pre-filled fluid chambers typically include a bung or stopper that covers an opening through the chamber wall and keeps fluid in the chamber until it is to be used. Fluid communication with the chamber can then be established by penetrating the bung or stopper with a hollow needle. For many well-known reasons, it is essential that the user be somehow protected from the needle as it is being manipulated to penetrate the stopper. Due to the unique physical features of a so-called “inverted syringe,” the establishment of fluid communication with a pre-filled fluid compartment may be particularly problematic.

In light of the above, it is an object of the present invention to provide a device that passively covers and protects a needle after first filling the device with medicament and then injecting the medicament into a patient. It is another object of the present invention to provide a device which guards the needle prior to an injection procedure and uses the same guard to passively guard the needle after an injection procedure. It is still another object of the present invention to provide a device in which the position of the needle guard is controlled and regulated by plunger movements that are required in a typical fill and inject procedure. It is yet another object of the present invention to provide a device having an integral mechanism that prevents reuse of the syringe (after use and contamination) by disabling the plunger at the completion of an injection procedure. Still another object of the present invention is to provide such a device for use with commercially available needles. Still another object of the invention is to provide a device with a needle guard that is movable to allow mounting of a needle on the device before use. An object of the present invention is to provide a safe and efficient fluid transfer apparatus for use with the pre-filled fluid compartment of an inverted syringe. Another object is to provide a fluid transfer apparatus that can effectively establish fluid communication with a fluid chamber when an access port to the chamber is in an effectively inaccessible location. Yet another object of the present invention is to provide a protective device for a medical syringe that is easy to use, relatively simple to implement, and comparatively cost effective.

### SUMMARY OF THE INVENTION

A device for expelling a fluid, such as a medicament, through a hypodermic needle mounted on a hub includes an extended luer member that has a proximal portion, a distal portion and a fluid conduit extending along an axis therebetween. The distal portion of the extended luer member is dimensioned to engage the needle hub to provide fluid communication between the fluid conduit and the needle. When engaged, the needle extends away from the distal portion of the luer member to a sharp needle tip at its own distal end. Additionally, the device includes an adapter for anchoring the proximal portion of the luer member. The adapter includes a substantially cylindrical-shaped wall surrounding a cavity bounded by an open distal end and a proximal end substantially covered by a base. Slidingly mounted on the adapter is a cylindrical-shaped inverted plunger that is movable thereon between an advanced position and a withdrawn position. When the plunger is in the withdrawn position, a fluid chamber is created between the plunger and the adapter base. Specifically, the chamber is formed by a seal engaging the proximal portion of the luer member which is slidingly received by the plunger to form a fluid tight boundary for the fluid chamber. For the present invention, a tube-shaped needle guard having a lumen is biased by a biasing member such as a spring to extend distally from the distal portion of the luer member when the plunger is in the advanced position. The guard is selectively engageable with the plunger to be retracted into the adapter cavity and over the elongated luer member to expose the distal portion of the luer member for fluid engagement with the needle hub when the plunger is moved to the withdrawn position.

In greater structural detail, the luer member includes a head section that engages the adapter. The head section is connected to a shaft section which extends distally from the head section along the axis to a shaft end that is spaced from the head section by a shaft length that is at least as long as the length of the adapter. Therefore, the luer member extends through the cavity and the open distal end of the adapter. As the extremity of the distal portion of the luer member, the shaft end is dimensioned for engagement with the needle hub. Engagement between the needle hub and shaft end may be achieved through a number of known methods. For instance, the needle hub may include a circumferential protrusion that fits into a corresponding circular groove on the shaft end. Alternatively, the needle hub may include male or female threadings to allow the hub to be screwed into reciprocal threadings on the shaft end. Or, more simply, the needle hub may be slipped snugly onto the shaft end.

As described in greater detail below, a mechanism provides selective engagement between the plunger and guard during the course of an injection procedure. More specifically, the plunger movements that are required to expose the distal portion of the extended luer member for mounting a needle thereon, to fill the fluid chamber, and to dispense a fluid from the fluid chamber also function to control the position of the guard. In functional overview, prior to an injection procedure, the guard is locked in an extended position distal of the luer member and can only be unlocked by a movement of the plunger. Once unlocked, the guard can be retracted to expose the distal portion of the luer member. This allows the needle hub to be mounted on the distal portion of the luer member, and in addition, allows the needle to be inserted into a medicament vial to fill the fluid chamber and to be inserted into a patient for an injection. When the plunger is withdrawn proximally relative to the adapter to create the fluid chamber, the plunger and guard engage one another, and the guard moves proximally to expose the distal portion of the luer member and a needle mounted thereon. On the other hand, when the plunger is advanced (i.e. moved distally), the plunger releases the guard. Once released, the guard is free to move distally under the influence of the spring. As a consequence of this interaction, after the plunger is depressed to complete an injection, the guard is released and allowed to move distally to its extended position to cover and protect the needle.

In operation, the plunger is initially located in an advanced position relative to the adapter. Next, the plunger is withdrawn proximally which causes the plunger to engage the guard and to move the guard proximally with the plunger to a retracted position. With the guard retracted, the next step is to mount the needle hub onto the exposed shaft end of the luer member. Then the distal tip of the needle may be inserted into a medicament vial. At this point, the plunger can be depressed to expel air into the vial and void the fluid chamber. During plunger advancement, the plunger operatively disengages the guard. Thus, distal movement of the guard is only prevented by the contact between the guard and the vial. Next, the plunger can be withdrawn to fill the fluid chamber with medicament. During this plunger withdrawal, the plunger again engages and retracts the guard. Thus, when the needle is removed from the vial, the distal tip of the needle remains unguarded and exposed. The syringe is now ready for an injection.

To inject a medicament into a patient, the distal tip of the needle is inserted into the patient and the plunger depressed. This distal advancement of the plunger releases the guard. Once released, the guard is free to move distally under the influence of the spring. Thus, as the needle is withdrawn from the patient, the needle retracts proximally into the guard, which remains in contact with the patient's skin. Once the syringe has been removed from the patient, the plunger and adapter can be advanced distally relative to the syringe body to lock the guard in place.

In accordance with another aspect of the present invention, a fluid transfer apparatus essentially includes a plunger assembly that is combined with a needle assembly. In combination, these assemblies interact with a pre-filled fluid chamber to transfer fluid from the container. Structurally, the container (vial) is basically a hollow, cylindrical-shaped tubular body that has two open ends. One end of the container (vial) is formed with an orifice that is covered by a fluid-tight stopper, while the other end is formed with an orifice for receiving the plunger assembly. Thus, a fluid chamber can be established between the stopper and the plunger assembly. As envisioned for the present invention, the fluid chamber of the container (vial) will be pre-filled, preferably with a fluid medicament. The plunger assembly can then be advanced into the chamber to expel fluid from the chamber.

In detail, the needle assembly of the fluid transfer apparatus includes a straight, doubled-ended, elongated hollow needle that has a sharpened first end, and a sharpened second end. It also includes a hub that is affixed to the needle at a distance “I” from its first end by a means well known in the pertinent art. Importantly, the hub is preferably formed with a pair of diametrically opposed flanges, each of which extends outwardly, in a radial direction from the needle. More flanges can be used, however, if desired. The needle assembly also includes a hollow tubular sleeve that has a lumen for receiving the needle. When joined with the needle, the sleeve covers the second end of the needle and extends from this second end, into contact with the hub. As discussed in greater detail below, prior to an operation of the fluid transfer apparatus of the present invention, the sleeve is used to activate the fluid transfer apparatus, and it is then removed from the needle.

Insofar as the plunger assembly is concerned, it includes a housing that is a generally hollow, cylindrical-shaped, tubular body having an inner wall that defines an interior. The plunger assembly also includes at least one detent that is formed on the inner wall of the housing, and is oriented to project into its interior. The detent(s) interacts with corresponding flanges on the hub and, accordingly, for a two flange hub, there are two diametrically opposed detents. Additionally, a bung is positioned at one end of the housing. Preferably, this bung is made of an elastomeric material so it can be penetrated by a needle. With this structure, when the plunger assembly and its bung are inserted into the open end of the container, the fluid chamber is established in the container between the stopper of the container and the bung of the plunger assembly.

As indicated above, the needle assembly of the fluid transfer apparatus interacts with both the container (vial) and the plunger assembly. For its interaction with the plunger assembly, the needle assembly is mounted in the interior of the housing for movement from a first position to a second position. More specifically, when the needle assembly is in the first position, the first end of the needle is against the bung at the end of the housing. Thus, the hub is positioned at the distance “I” from the bung. Also, while in this first position, the detents on the inner wall of the housing are intermediate the bung and the flange on the hub. As indicated above, the movement of the needle assembly in the housing, from its first position to its second position, is accomplished by pushing on the sleeve in a proximal direction along the longitudinal axis of the needle. This activates the fluid transfer apparatus.

During activation of the fluid transfer apparatus, flanges on the hub are initially in an unstressed condition and they each extend a radial distance “r₁” from the needle. This places the flanges in contact with the inner wall of the housing. As the needle assembly is moved axially through the distance “I”, and into the second position, each flange on the hub rides up and over a respective detent. As they do so, each flange withdraws to within a radial distance “r₂” from the needle, and is in a substantially stressed condition. After passing the detents, each flange again extends to the radial distance “r₁”, and the hub becomes fixedly held between the bung and the detents. In their relation to each other, “r₂” is less than “r₁”. When the needle assembly and hub is in the second position, however, (i.e. after the needle assembly has been advanced toward the bung) the first end of the needle penetrates the bung. This establishes fluid communication between the needle and fluid in the chamber for the transfer of fluid from the chamber. Further, and importantly, in the second position, the flange is in contact with the detent to fixedly hold the needle assembly in the second position.

In more detail, when the needle assembly is in its second position, the needle will penetrate a distance “d” through the bung. Thus, in order to establish fluid communication between the needle and fluid in the container (vial), “d” must be less than “I”. Moreover, it is preferable that the distance “I”-“d” be as small as possible. Furthermore, as stated above it is preferable for the bung to be made of an elastomeric material that can be easily penetrated by the needle, and it is preferable that the needle be made from a stainless steel hypotube. Regardless, with the needle assembly in its second position, the plunger assembly can be advanced into the fluid chamber of the container to expel fluid from the chamber, through the needle.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a perspective view of a device for expelling a fluid through a hypodermic needle mounted on a hub, shown in its initial configuration;

FIG. 2 is a perspective view of a hypodermic needle mounted on a hub for use with the device of FIG. 1;

FIG. 3A is a sectional view of the syringe as seen along line 3-3 in FIG. 1, shown after a needle has been mounted on the luer member and with the guard locked over the needle's distal tip;

FIG. 3B is a sectional view of the syringe as in FIG. 3A, shown after a plunger movement has unlocked and distally retracted the guard;

FIG. 3C is a sectional view of the syringe as in FIG. 3A, shown after the needle's distal tip has been inserted into an object (i.e. medicament vial or patient) and thereafter the plunger has been advanced proximally;

FIG. 3D is a sectional view of the syringe as in FIG. 3A, shown after the plunger and adapter have been advanced distally relative to the syringe body to lock the guard in position and prevent inadvertent reuse of the syringe;

FIG. 4A is a sectional view of the syringe as seen along line 4-4 in FIG. 1, shown after a needle has been mounted on the luer member and with the guard locked over the needle's distal tip;

FIG. 4B is a sectional view of the syringe as in FIG. 3A, shown after a plunger movement has unlocked and distally retracted the guard;

FIG. 4C is a sectional view of the syringe as in FIG. 3A, shown after the needle's distal tip has been inserted into an object (i.e. medicament vial or patient) and thereafter the plunger has been advanced proximally;

FIG. 4D is a sectional view of the syringe as in FIG. 3A, shown after the plunger and adapter have been advanced distally relative to the syringe body to lock the guard in position and prevent inadvertent reuse of the syringe;

FIG. 5 is a perspective view of a fluid transfer apparatus;

FIG. 6A is a cross-sectional view of the apparatus as seen along the line 6-6 in FIG. 5 in a configuration prior to its activation;

FIG. 6B is a cross-sectional view of the apparatus as seen along the line 6-6 in FIG. 5 in a configuration subsequent to its activation; and

FIG. 6C is a cross-sectional view of the apparatus as seen along the line 6-6 in FIG. 5 after a fluid transfer operation.

### DESCRIPTION OF THE PREFERRED EMBODIMENTS

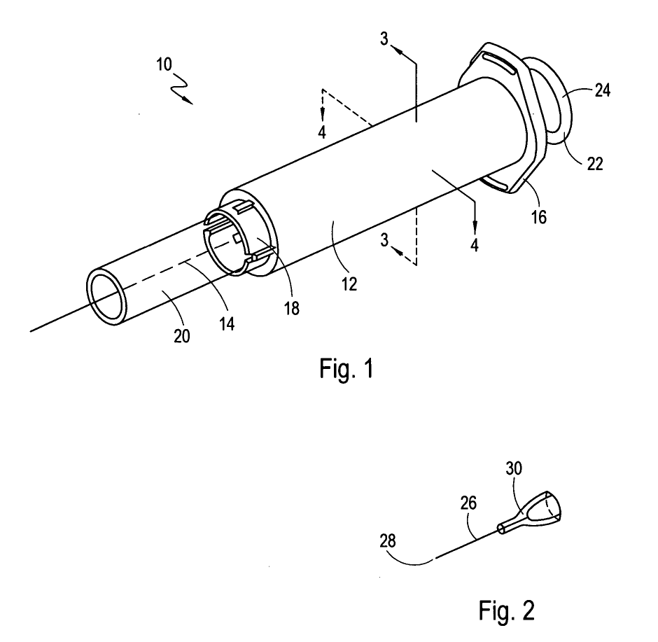

Referring initially to FIG. 1, a syringe device for expelling a fluid through a needle mounted on a hub is shown and generally designated 10. As shown in FIG. 1, the device 10 includes a substantially cylindrical syringe body 12 that is centered on an axis 14 and formed with a finger grip 16 at its proximal end. FIG. 1 further shows that the device 10 includes an adapter 18 sized to fit within the syringe body 12. The adapter 18 includes a cylindrical portion that is also centered on the axis 14. For the device 10, a substantially cylindrical needle guard 20 is provided and positioned co-axially with both the syringe body 12 and adapter 18. The guard 20 is sized to fit within the adapter 18. It can be further seen that the device 10 includes a plunger 22 that is formed with a grip flange 24 at its proximal end.

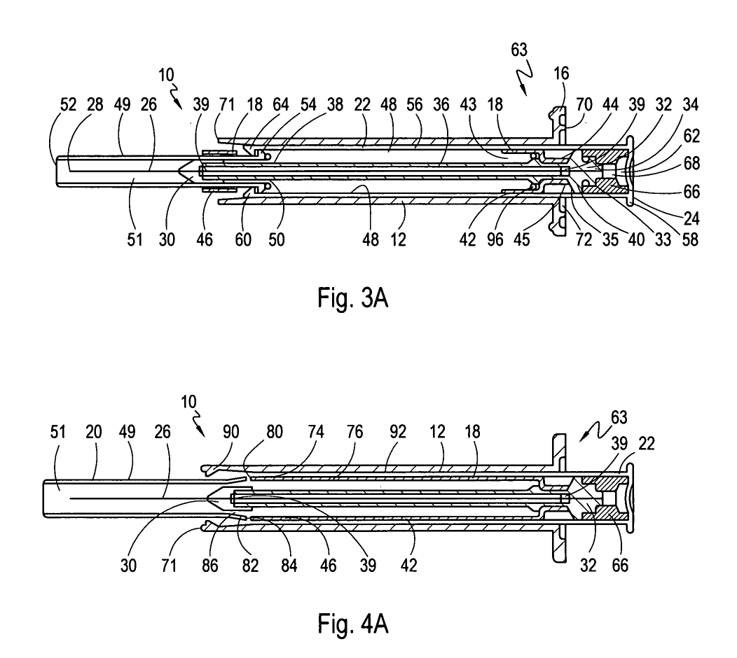

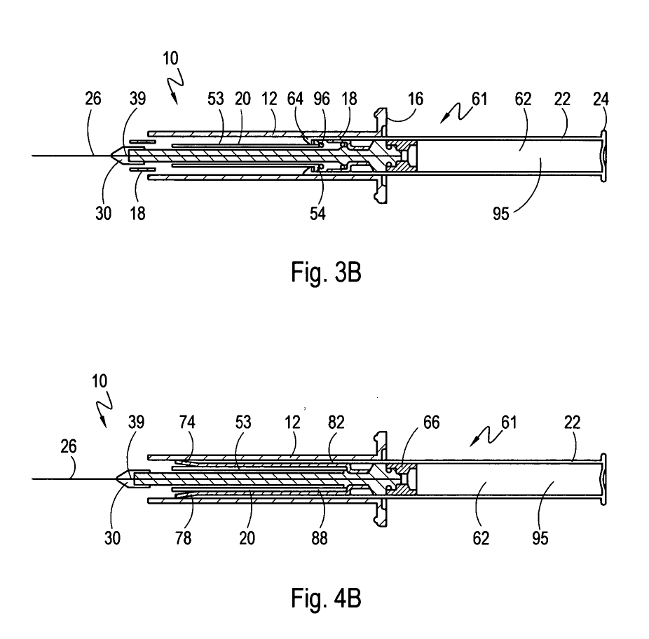

Referring to FIG. 2, a straight, elongated hypodermic needle 26 is shown extending from a sharp needle tip 28 to a needle hub 30. As best seen in FIG. 3A, the needle 26 may be mounted to the passively guarded, fillable injection device 10. Specifically, the device 10 includes a luer member 32 that receives and engages the needle hub 30. The luer member 32 has a proximal portion or head 34. Extending distally from the head 34 is a substantially cylindrical shaft 36 centered on the axis 14. The needle hub 30 is mounted to the luer member 32 at the shaft's distal portion or distal shaft end 38. Additionally, the head 34 has a proximal side 33 and a distal side 35 that engages the adapter 18. Circumferentially-spaced truss-like webs 40 are provided on the luer member 32 to reinforce the connection between the shaft 36 and the head 34. Furthermore, the luer member 32 includes a pipe-like conduit 39 that extends from the proximal side 33 of the head 34 to the shaft end 38. When the needle hub 30 is frictionally mounted on the shaft end 38, the needle hub 30 and luer member 32 are sealed together to establish fluid communication between the needle 26 and the conduit 39.

As shown in FIG. 3A, the adapter 18 engages the luer member 32 about the webs 40 thereby preventing rotational movement therebetween. The adapter 18 includes a substantially cylindrical wall 42 that is centered on the axis 14 and forms a cavity 43. The wall 42 extends from a proximal end 44 substantially covered by a base 45 to an open distal end 46. At its proximal end 44, the adapter 18 has a narrow circumference and is designed to engage the distal side 35 of the head 34 of the luer member 32. At its distal end 46, the adapter 18 has a broad circumference and is designed to engage the plunger 22 and receive the guard 20. As can be seen in FIG. 3A, the adapter 18 also includes two oppositely positioned, axially aligned slits 48.

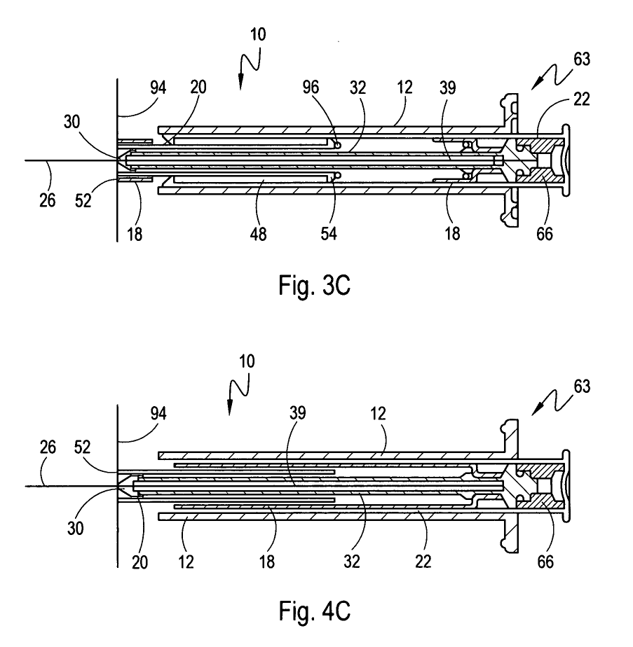

As best seen in FIG. 3A, the adapter 18 is sized to allow the cylindrical guard 20 to move along the axis 14 into and out of the adapter cavity 43. Specifically, the guard 20 may be moved between an extended position 49 (shown in FIGS. 3A and 4A) to a retracted position 53 (shown in FIGS. 3B and 4B). Structurally, the guard 20 is a shell forming a lumen 51 that extends between an open proximal end 50 and an open distal end 52. The guard 20 includes abutments 54 that extend radially outward from the proximal end 50.

Also in FIG. 3A, it can be seen that the inverted plunger 22 has a substantially cylindrical side member 56 that extends from a closed proximal plate member 58 to an open distal edge 60. Structurally, the cylindrical side member 56 surrounds a fluid chamber 62 and is slidingly mounted on the proximal end 44 of the adapter 18. FIG. 3A further shows that the plunger 22 is formed with tangs 64 that extend radially inward (i.e. toward the axis 14) and distally from the cylindrical side member 56 of the plunger 22.

It can be seen in FIG. 3A, that the device 10 includes an elastomeric seal 66 that is attached onto the proximal end 34 of the luer member 32. Specifically, the seal 66 is press fitted onto the proximal end 34 of the luer member 32. As shown, the seal 66 has a generally fusiform or spindle-like shape and is formed with a through-hole 68. When the open distal edge 60 of the plunger 22 is slid over the luer member 32 and adapter 18, the seal 66 compresses between the luer member 32 and the cylindrical side member 56 of the plunger 22 to establish sealed fluid communication between the fluid chamber 62 and the conduit 39 of the luer member 32. When the needle hub 30 is mounted on the luer member 32 to establish fluid communication between the needle 26 and the conduit 39, the plunger 22 can be moved to a withdrawn position 61 (shown in FIGS. 3B and 4B) to draw fluid through the needle 26 and into the chamber 62. Furthermore, the plunger 22 can be moved to an advanced position 63 (shown in FIGS. 3C and 4C) to expel fluid from the chamber 62 through the needle tip 28.

As further shown in FIG. 3A, the syringe body 12 extends from an open proximal end 70 to an open distal end 71. Positioned at the proximal end 70, the finger grip 16 includes a recess 72 sized to receive the grip flange 24 of the plunger 22. Functionally, the plunger 22 can be advanced distally after an injection until the grip flange 24 is positioned in the recess 72. Once the grip flange 24 is positioned in the recess 72 it cannot be removed; therefore, subsequent movement of the plunger 22 relative to the syringe body 12 is effectively prevented.

Turning to FIG. 4A, other features of the device 10 may be seen. Specifically, the adapter 18 is shown having cam levers 74 positioned at its distal end 46. The cam levers 74 are in a biased position 76 in which the plunger 22 forces them to be coincident with the rest of the cylindrical wall 42. However, the cam levers 74 mechanically prefer a relaxed position 78 (shown in FIG. 4B) in which the distal ends 80 of the cam lever 74 extend radially outward from the cylindrical wall 42.

As shown in FIG. 4A, the guard 20 has hinges 82. Similar to the cam levers 74 of the adapter 18, the hinges 82 of the guard 20 have a relaxed position 84 in which the proximal ends 86 of the hinges 82 extend radially outward from the rest of the guard 20. The biased position 88 of the hinges 82 is shown in FIG. 4B.

As further shown in FIG. 4A, the syringe body 12 can also include flanges 90 at its distal end 71. The flanges 90 extend distally and radially inward from the cylindrical portion 92 of the syringe body 12. Their purpose is discussed below.

By cross-referencing FIGS. 3A-D and 4A-D, it can be seen that the device 10 includes a mechanism to lock the guard 20 in an extended position 49 covering the needle tip 28 prior to an injection procedure. Once locked, the guard 20 can only be unlocked by movement of the plunger 22. As previously discussed, the adapter 18 is formed with cam levers 74 having distal lever ends 80. Comparing FIG. 4A with FIG. 4B, it can be seen that the cam levers 74 are deflectable by the cylindrical side member 56 of the plunger 22 from a relaxed position 78 (FIG. 4B) to a biased position 76 (FIG. 4A). In the relaxed position 78 (FIG. 4B), the cam levers 74 extend radially outward from the remaining cylindrical section of the adapter 18. On the other hand, as shown in FIG. 3A, in the biased (i.e. deflected) position 76, the cam levers 74 are coincident with the remaining cylindrical wall 42 of the adapter 18. When the plunger 22 is in the advanced position 63 shown in FIG. 4A, the cylindrical side member 56 of the plunger 22 contacts the cam levers 74 and deflects them into the biased position 76. As shown in FIG. 4A, when the cam levers 74 are in the biased position 76, the lever ends 80 engage the proximal ends 86 of the hinges 82 of the guard 20 and prevents proximal movement of the guard 20. When the plunger 22 is in its withdrawn position 61 as shown in FIGS. 3B and 4B, the cam lever 74 relaxes into its undeflected, outward position 78 (as shown in FIG. 4B) and allows the guard 20 to move proximally.

As an additional locking mechanism, the syringe body 12 may be moved relative to the adapter 18 to deflect the cam levers 74 of the adapter 18 with its flanges 90. As shown in FIG. 4D, the flanges 90 are moved toward the adapter 18 when the plunger 22 and adapter 18 are fully pushed into the syringe body 12. As a result, the flanges 90 contact and force the cam levers 74 of the adapter 18 to the biased position 76 to lock the device 10 to prevent any further proximal movement of the needle guard 20. This prevents inadvertent reuse of the device 10.

### Operation

Initially, the device 10 is provided without a needle 26. To mount a needle 26 on the device 10, the needle guard 20 is first moved to the retracted position 53 by withdrawing the plunger 22. Then the needle hub 30 is frictionally engaged with the shaft end 38 of the luer member 32 as can be understood from FIGS. 3B and 4B. After mounting the needle hub 30 on the shaft end 38, the needle guard 20 is allowed to move to its extended position 49 to cover the needle 26 by moving the plunger 22 to its advanced position 63 as shown in FIG. 3A. As further shown in FIG. 3A the tangs 64 of the plunger 22 extend through the slits 48 in the adapter 18 to engage the abutments 54 and retract the guard 20 when the plunger 22 is withdrawn.

From FIG. 4A, it can be seen that the cylindrical side member 56 of the plunger 22 holds the cam levers 74 deflected inward to lock the guard 20 and prevent proximal movement of the guard 20. As illustrated by FIGS. 3A-B and 4A-B, use of the device 10 begins by withdrawing the plunger 22. Such proximal movement of the plunger 22 has several effects. Specifically, as shown in FIGS. 4A and 4B, initial proximal movement of the plunger 22 allows the cam levers 74 to relax outwardly from the axis 14 and unlock the guard 20 for proximal movement. As shown in FIGS. 3A and 3B, additional proximal movement of the plunger 22 engages the tangs 64 with the abutments 54, causing the guard 20 to be retracted with the plunger 22. Also, withdrawal of the plunger 22 draws air (or other fluid) through the needle 26 and into the fluid chamber 62.

Once the guard 20 has been retracted as shown in FIGS. 3B and 4B, the next step is to insert the exposed distal needle tip 28 into a medicament vial (illustrated by surface 94 in FIGS. 3C and 4C). At this point, the plunger 22 can be depressed as shown in FIGS. 3C and 4C to expel air into the vial and void the fluid chamber 62. Comparing FIGS. 3B and 4B with FIGS. 3C and 4C, it can be seen that during its advance the plunger 22 disengages the guard 20. Thus, as illustrated by FIGS. 3C and 4C, after advancing the plunger 22, distal movement of the guard 20 is only prevented by the contact between the distal end 52 of the guard 20 and the surface 94. Next, the plunger 22 can be withdrawn to fill the chamber 62 with medicament fluid 95 (note FIGS. 3B and 4B are representative of the configuration of the device 10 after the chamber 62 is filled with medicament 95). From FIG. 3B, it can be seen that during withdrawal of the plunger 22, the tangs 64 reengage the abutments 54. The result is that the plunger 22 engages the guard 20 and prevents distal advancement of the guard 20. As illustrated by FIGS. 3B and 4B, when the needle 26 is removed from the vial, the distal tip 28 of the needle 26 remains unguarded and exposed. The device 10 is now ready for an injection.

As illustrated by FIGS. 3C and 4C, to inject a medicament into a patient, the distal tip 28 of the needle 26 is inserted into the patient (represented by surface 94) and the plunger 22 is depressed. As shown in FIG. 3C, the distal advancement of the plunger 22 releases the guard 20. Once released, the guard 20 is free to move distally under the influence of a coil spring 96 that is interposed between the guard 20 and the adapter 18. Thus, as the needle 26 is withdrawn from the patient, the needle 26 retracts proximally into the guard 20 which remains in contact with the patient's skin (represented by surface 94). FIGS. 3A and 4A are representative of the device 10 after the needle 26 has been withdrawn from the patient and the needle 26 has passively retracted into the guard 20.

Once the device 10 has been removed from the patient, the plunger 22 and the adapter 18 can be advanced distally relative to the syringe body 12 to lock the guard 20 in place (FIG. 4D). FIGS. 3D and 4D also show that this places the grip flange 24 of the plunger 22 in the recess 72 formed in the syringe body 12. Functionally, once the device 10 is in the configuration shown in FIGS. 3D and 4D, the plunger 22 is disabled and the guard 20 completely covers the hollow needle 26 to protect the user from unwanted needle sticks and prevents inadvertent reuse of the device 10.

### Additional Aspects

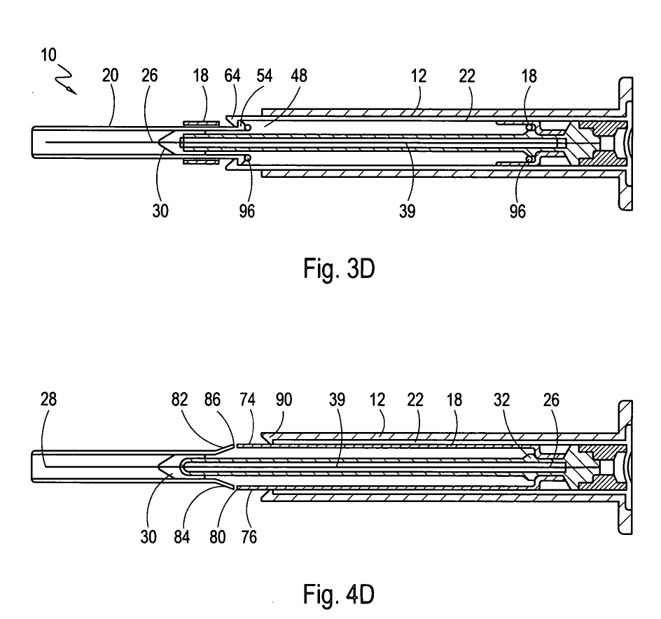

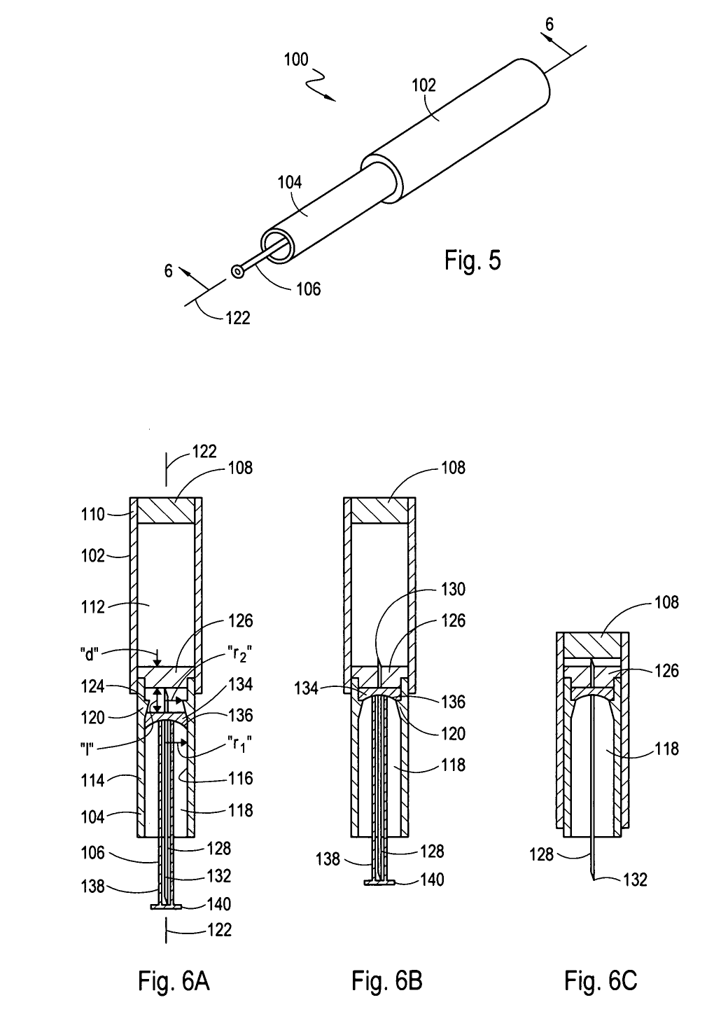

Turning now to FIG. 5, a fluid transfer apparatus that can be used individually, or in combination with the syringe guard disclosed above, is shown and is generally designated 100. In FIG. 5 it will be seen that the apparatus 100 includes a fluid container (vial) 102, a plunger assembly 104, and a needle assembly 106. As disclosed in more detail below, it will be appreciated that the apparatus 100 is activated for a fluid transfer operation by first engaging the needle assembly 106 with the plunger assembly 104. In combination, the needle assembly 106 and the plunger assembly 104 are then advanced into the container 102 to expel fluid from the container 102. The structural details of apparatus 100 will, perhaps, be best appreciated with reference to FIG. 6A.

In FIG. 6A it will be seen that the fluid container 102 includes a stopper 108 that is positioned at the end 110 of container 102 to help define a fluid chamber 112. As intended for the present invention, after a fluid has been pre-filled into the chamber 112, the stopper 108 can be positioned, as shown. After being so positioned, however, it is important that the stopper 108 remain fixedly in place on the container (vial) 102 during a subsequent operation of the apparatus 100.

Still referring to FIG. 6A, the plunger assembly 104 is shown to include a housing 114 that has an inner wall 116 which defines an interior 118 for the housing 114. FIG. 6A also shows that at least one detent 120 is formed on the inner wall 116, and that the detent 120 is formed with a seat 124 that extends radially inward toward the longitudinal axis 122 of the apparatus 100. Also, the plunger assembly 104 includes a bung 126 that has a depth distance “d,” and covers one end of the housing 114. As shown, the bung 126 is specifically positioned to create a fluid tight seal for the chamber 112, and for subsequent advancement into the chamber 112. The fluid chamber 112 of container 102 is thereby established between the stopper 108 of container (vial) 102, and the bung 126 of the plunger assembly 104. For purposes of the present invention, the bung 126 is preferably made of an elastomeric material that can be penetrated by a sharp object to establish fluid communication with fluid in the chamber 112.

The needle assembly 106 of the apparatus 100 will be best appreciated by cross-referencing FIG. 6A with FIG. 6B. In detail, the needle assembly 106 includes an elongated, double-ended needle 128 having a sharp end 130 (see FIG. 6B) and an opposite sharp end 132. Preferably, the needle 128 is made from a hollow stainless steel hypotube. Additionally, the needle assembly 106 includes a hub 134 that is fixedly attached to the needle 128 at a distance “I” from the end 130. Attachment of the hub 134 to the needle 128 can be accomplished by any method well known in the pertinent art. Further, the needle assembly 106 includes an actuator sleeve 138 that is positioned over the needle 128. Importantly, the actuator sleeve 138 covers the needle 128 from its end 132 to the hub 134, and is in contact with the hub 134.

In the operation of the apparatus 100 of the present invention, a user (not shown) pushes against an abutment 140 that is formed at the distal end of the actuator sleeve 138. This causes the actuator sleeve 138 to urge against the hub 134, and to move the entire needle assembly 106 in a proximal direction along the axis 122. The consequence of this is that the needle assembly 106 moves through the distance “I” from a first position (see FIG. 6A), into a second position (see FIG. 6B). Also, during this movement, the sharp end 130 of needle 128 penetrates the bung 126 through its distance “d” (i.e. “I”>“d”) to establish fluid communication between fluid in the chamber 112 and the hollow needle 128. As this is happening, the needle assembly 106 becomes fixedly engaged with the plunger assembly 104.

Engagement of the needle assembly 106 with the plunger assembly 104 occurs due to the interaction of the flanges 136 on hub 134 with the detent 120 on housing 114. Specifically, as the hub 134 is advanced with the needle assembly 106 from its first position, and into its second position, the flanges 136 ride up over the detent 120. As they do so, the flanges 136 go from an unstressed condition wherein they extend a radial distance “r₁” from the axis 122 (see FIG. 6A), to a stressed condition at the detent 120, wherein they withdraw to a radial distance “r₂” from the axis 122. This occurs before the hub 134 reaches the second position. After the hub 134 has been advanced beyond the detent 120, and into its second position, the flanges 136 again extend the distance “r₁” from the axis 122. Also, they are, again, in an unstressed condition. Further, in the second position, the flanges 136 are in contact with the seat 124 of the detent 120. Thus, in the second position, the hub 134 abuts the bung 126 and the flanges 136 abut the detent 120 to fixedly hold the needle assembly 106 on the plunger assembly 104.

Once the needle assembly 106 has been fixedly engaged with the plunger assembly 104, the actuator sleeve 138 is removed from the needle 128. This exposes the end 132 of the needle 128. The apparatus 100 is now ready to expel fluid from the chamber 112, for such purposes as an injection of fluid medicament into a patient (not shown). Specifically, this task is accomplished by advancing the combination needle assembly 106 and plunger assembly 104 into the chamber 112. The result of this advancement is to expel fluid from the fluid chamber 112 through the needle 128, and to continue doing so, until the apparatus 100 is in the configuration shown in FIG. 6C.

While the particular devices and methods as herein shown and disclosed in detail are fully capable of obtaining the objects and providing the advantages herein before stated, it is to be understood that they are merely illustrative of the presently preferred embodiments of the invention and that no limitations are intended to the details of construction or design herein shown other than as described in the appended claims.

What is claimed is:

1. A fluid transfer apparatus which comprises:
a vial having a chamber filled with the fluid;
an elongated hollow needle having a first end and a second end and defining an axis;
a hub affixed to said needle at a distance “I” from the first end of said needle, said hub being formed with at least one flange, wherein the flange extends in a radial direction from the needle;
a housing formed with a detent;
a bung mounted on said housing, with said housing being engageable with said hub for movement of said needle with said hub from a first position to a second position, wherein with the hub in the first position, the first end of the needle is against said bung and the detent on said housing is intermediate said bung and the flange, and wherein with the hub in the second position, the first end of the needle penetrates said bung to establish fluid communication between the needle and fluid in the chamber for fluid transfer, and further wherein the flange is in contact with the detent to fixedly hold the needle in the second position; and
a means for advancing said housing into said chamber to expel fluid therefrom through said needle, when said needle is in the second position.

2. An apparatus as recited in claim 1 wherein said vial is formed with an orifice and said apparatus further comprises a stopper positioned in the orifice of said vial to hold fluid in the chamber.

3. An apparatus as recited in claim 1 wherein said hub comprises a plurality of flanges.

4. An apparatus as recited in claim 1 wherein the flange is in an unstressed condition and extends a radial distance “r₁” from said needle when said needle is in the first position and in the second position, and the flange is in a substantially stressed condition and withdraws to a radial distance “r₂” from said needle when passing over the detent during movement from the first position to the second position, and further wherein “r₂” is less than r₁”.

5. An apparatus as recited in claim 1 wherein the fluid is a medicament.

6. An apparatus as recited in claim 1 further comprising a hollow sleeve positioned to cover the needle from the hub to the second end of the needle, said sleeve being positioned to urge against said hub for movement thereof from the first position to the second position, and wherein said sleeve is removable from the needle when the needle and said hub are in the second position.

7. An apparatus as recited in claim 1 wherein the needle penetrates said bung through a distance “d”, and “d” is less than “I”.

8. An apparatus as recited in claim 1 wherein said bung is made of an elastomeric material.

9. An apparatus as recited in claim 1 wherein said needle is made from a stainless steel hypotube.

10. A fluid transfer apparatus which comprises:
a container for holding the fluid, said container having an orifice;
a penetrable bung covering the orifice;
a needle assembly including an elongated hollow needle having a first end and a second end;
a means for supporting said needle assembly for movement thereof from a first position to a second position, wherein said supporting means holds the first end of the needle against the bung when said needle assembly is in the first position; and
a means for urging the needle assembly from the first position into the second position to penetrate the bung with the needle and to contact said supporting means with said needle assembly to fixedly hold the needle assembly in the second position and establish fluid communication between said container and the needle for transfer of fluid from said container.

11. An apparatus as recited in claim 10 wherein said needle assembly further comprises a hub affixed to said needle at a distance “I” from the first end of said needle, said hub being formed with at least one flange, wherein the flange extends in a radial direction from the needle.

12. An apparatus as recited in claim 11 wherein the flange is in an unstressed condition and extends a radial distance “r₁” from said needle when said needle is in the first position and in the second position, and the flange is in a substantially stressed condition and withdraws to a radial distance “r₂” from said needle when passing over the detent during movement from the first position to the second position, and further wherein “r₂” is less than r₁”.

13. An apparatus as recited in claim 11 further comprising a hollow sleeve positioned to cover the needle from the hub to the second end of the needle, said sleeve being positioned to urge against said hub for movement thereof from the first position to the second position, and wherein said sleeve is removable from the needle when the needle and said hub are in the second position.

14. An apparatus as recited in claim 11 wherein the needle penetrates said bung through a distance “d”, and “d” is less than “I”.

15. An apparatus as recited in claim 11 wherein said bung is mounted on a plunger assembly and wherein said plunger assembly is mounted on said container for advancement through said container to expel fluid therefrom and through said needle, when said needle is in the second position.

16. A method for transferring fluid which comprises the steps of:
providing a container for holding the fluid, said container having an orifice;
positioning a plunger assembly in the orifice of the container for advancement of the plunger assembly into the container, the plunger assembly including a penetrable bung to cover the orifice of the container;
supporting a needle assembly for movement thereof from a first position to a second position, wherein the needle assembly includes an elongated hollow needle having a first end and a second end and the first end of the needle is held against the bung when the needle assembly is in the first position; and
urging the needle assembly from the first position into the second position to penetrate the bung with the needle and to contact the supporting means with the needle assembly to fixedly hold the needle assembly in the second position and establish fluid communication between the container and the needle for transfer of fluid from the container.

17. A method as recited in claim 16 wherein the needle assembly further comprises a hub affixed to the needle at a distance “I” from the first end of the needle and is formed with at least one flange, wherein the flange is in an unstressed condition and extends a radial distance “r₁” from said needle when said needle is in the first position and in the second position, and the flange is in a substantially stressed condition and withdraws to a radial distance “r₂” from said needle when passing over the detent during movement from the first position to the second position, and further wherein “r₂” is less than r₁”.

18. A method as recited in claim 17 wherein said urging step comprises the steps of:
using a hollow sleeve, wherein the sleeve is positioned to cover the needle from the hub to the second end of the needle, to urge against the hub for movement thereof from the first position to the second position; and
removing the sleeve from the needle when the needle and the hub are in the second position.

19. A method as recited in claim 18 wherein the needle penetrates said bung through a distance “d”, and “d” is less than “I”.

20. A method as recited in claim 19 further comprising the step of advancing a plunger through the container to expel fluid therefrom and through the needle, when the needle in is the second position.
